(19) United States
(12) Patent Application Publication
(10) Pub. No.: US 2008/0228124 A1
(43) Pub. Date: Sep. 18, 2008

(54) RADIOGRAPHIC CONTRAST MEDIA CAPTURE MATERIAL

(75) Inventors: Gregory Lamps, Smyrna, GA (US); Jay Yadav, Hunting Valley, OH (US); Boris Mizaikoff, Ulm (DE)

Correspondence Address: THOMPSON HINE L.L.P.;Intellectual Property Group, P.O. BOX 8801, DAYTON, OH, 45401-8801, US

(73) Assignee: ATLA HOLDINGS, LLC, Atlanta, GA (US)

(21) Appl. No.: 12/031,996

(22) Filed: Feb. 15, 2008

Related U.S. Application Data
(60) Provisional application No. 60/901,260, filed on Feb. 15, 2007.

(51) Int. Cl. A61M 37/00

(52) U.S. Cl. 604/5.04; 604/85

(57) ABSTRACT

The present invention relates to materials, coatings, apparatus, and methods for removing radiocontrast agent from a patient. In accordance with one aspect of the invention, molecular imprinting technologies are utilized to create a safe, predictable and reliable medium for removal of nephrotoxic contrast agent from body fluids of a patient, such as the bloodstream. In accordance with certain aspects of the present invention, the contrast agent can be removed without mechanical filtering or any other mechanically damaging technique affecting the blood matrix. The molecularly imprinted medium (MIM) can be used to extract the contrast agent from the blood either intra- or extracorporeally.

### CROSS REFERENCE TO RELATED APPLICATION

This application claims the benefit of U.S. Provisional Application Ser. No. 60/901,260, filed Feb. 15, 2007, the contents of which are hereby incorporated by reference.

### BACKGROUND OF THE INVENTION

The present invention relates to methods and devices for removing radiocontrast agent from a patient using a molecularly imprinted medium (MIM). The invention further relates to materials and coatings containing the MIM's, as well as the methods and devices for producing the MIM's and coatings containing same.

Many medical procedures require the use of a radiographic contrast agent for accurate imaging. One common use of radiocontrast agents is for imaging the heart and surrounding vasculature. The contrast media is injected into the coronary arteries. After flowing through the cardiac arteries and veins, the mixture of the blood and contrast media flows through the coronary sinus prior to entering the right atrium of the heart and being circulated throughout the body.

For most patients, state of the art contrast media is easily filtered by the body from the blood stream, and excreted in the urine without causing damage. However, in some patients, especially those with pre-existing renal insufficiency, pre-existing diabetes, or reduced intravascular volume, the contrast presents a high likelihood of causing irreversible kidney damage. This damage to the kidneys is called contrast induced nephropathy (CIN). More particularly, CIN is acute renal failure that develops after administration of an iodinated radiocontrast agent. CIN leads to longer hospital stays, increased cost of care, and a higher incidence of mortality.

Current best methods to prevent CIN rely on prophylactic measures, e.g., the use of low-molarity or iso-molar (to blood) contrast media, and limited doses of contrast media in conjunction with hydration regimes for at-risk patients. However, prophylactic measures studied to date have shown imperfect results with some measures showing a beneficial result for some patients, but no universally successful treatment modality has been demonstrated.

Consequently, the reduction of the incidence of CIN by removal of the contrast agent is desirable. A procedure capable of removing the contrast agent would enable physicians to perform procedures that require contrast agent on patients who are at risk of CIN due to pre-existing renal insufficiency. The ability to selectively remove contrast agent from the bloodstream would also allow injection of a greater amounts of contrast agent into patients, thereby enabling more complex procedures. Additionally, it would reduce the amount of time the patient needs to be hospitalized or observed after administration of contrast media to screen for CIN. A reduction in the incidence of CIN would furthermore reduce costs for the patient and hospital, potentially shorten the duration of hospitalization, decrease incidence of mortality, and increase the quality of life for patients.

Methods which have been studied or proposed to remove contrast agent from the blood include centrifuging, and partial or complete filtration, e.g., by dialysis. Dialysis is a method to partially protect the kidneys, but suffers limitation due to damage caused to the blood matrix and separation of blood constituents, which should preferably remain in the blood. Centrifugation procedures suffer from similar limitations as dialysis.

Thus a need exists for devices and methods to remove radiocontrast agent from the blood of at-risk populations safely, predictably, selectively, and reliably.

### SUMMARY OF THE INVENTION

Stated generally, the present invention relates to materials, coatings, apparatus, and methods for removing radiocontrast agent from a patient, typically from the patient's bloodstream or other body fluid. The present invention further provides manufacturing methods for the matrix and corresponding apparatus.

The present invention utilizes molecular imprinting technologies to create a safe, predictable and reliable medium for removal of nephrotoxic contrast agent from a patient's body fluid, such as the bloodstream. In accordance with certain aspects of the present invention, the contrast agent can be removed without mechanical filtering or any other mechanically damaging technique affecting the blood matrix. The molecularly imprinted medium (MIM) can be used to extract the contrast agent from the blood either intra- or extracorporeally.

In accordance with another aspect of the present invention, a device for removing radiocontrast agent from a patient is described. The device includes a molecularly imprinted medium (MIM), wherein the MIM reversibly binds to a radiocontrast agent and optionally to a support. The support may be formed from the MIM or from any other materials compatible with the proposed use of the device.

### BRIEF DESCRIPTION OF THE DRAWINGS

FIG. 1 is a schematic illustrating the principle of molecular imprinting; and

FIG. 2 is a schematic representation of imprinting and removal procedures in molecularly imprinted hydrogels.

### DESCRIPTION OF THE INVENTION

The term “comprising” encompasses the more restrictive terms “consisting essentially of” and “consisting of”

All percentages, ratios and proportions used herein are by weight unless otherwise specified.

The term “MIM” as used herein refers to a molecularly imprinted medium. These materials are typically matrices with binding sites for specific molecules based on a combination of recognition mechanisms including size, shape, and functionality. The basic matrix of MIMs may be but is not limited to a polymer, a sol-gel, a hydrogel, a composite, or any other structural matrix capable of forming binding pockets within the material and/or at its surface that can selectively capture the template molecule.

Molecularly imprinted media are described in the following patents and publications, the relevant contents of which are incorporated herein by reference: U.S. Pat. No. 5,110,833 to Mosbach; U.S. Pat. No. 5,821,311 to Mosbach et al; U.S. Pat. No. 5,858,296 to Domb; U.S. Pat. No. 5,872,198 to Mosbach et al.; U.S. Pat. No. 6,638,498 to Green et al.; Mosbach, K. et al, “The Emerging Technique of Molecular imprinting and Its Future Impact on Biotechnology”, Biotechnology, vol Feb. 14, 1996, pp 163-170; G. Wulff. “Molecular Imprinting in Cross-Linked Materials with the Aid of Molecular Templates—A Way towards Artificial Antibodies” Angew. Chem. Intl. Ed. Engl., 34, 1812-1832 (1995); P. Hollinger, et al., “Mimicking Nature and Beyond” Trends in Biochemistry, 13(1), 79 (1995); Haupt, K., Mosbach, K. Trends Biotech, 16, 468-475 (1997); Davis et al, “Rational Catalyst Design via Imprinted Nanostructured Materials” Chem. Mater. 8 (1996) pp 1820-1839. and Wulff. G. et al, “Enzyme models Based on Molecularly Imprinted Polymers with Strong Esterase Activity” Angew. Chem. Int. Ed. Engl., 36 1962 (1997).

The present invention relates to methods and devices for removing radiocontrast agent from a patient using a molecularly imprinted medium (MIM). The invention further relates to materials and coatings containing the MIM's as well as the methods and devices for producing the MIM's and coatings containing same.

The MIM is capable of selectively capturing the radiocontrast agent, thereby facilitating removal of the radiocontrast agent from the patient's body. In accordance with certain aspects of the present invention, a method is provided for preventing or treating a patient suffering from contrast induced nephropathy.

The MIM can be used to extract the contrast agent from the blood either intra- or extracorporeally. If intracorporeal methods are utilized, the MIM can be brought into contact with the contrast agent in the blood in any of a number of ways. For example, the MIM can be configured directly as a surface coating or as a component of a surface coating on a catheter or other device, which is intraluminally inserted into the bloodstream. Alternatively, the MIM could form the structure of the device or a part of the device as well. This device can be guided to an appropriate site in the body, e.g., the superior vena cava, the inferior vena cava, the coronary sinus, or the portion of the aorta above the kidneys.

In accordance with certain aspects of the present invention, an extracorporeal extraction method is provided. The method would typically involve drawing the blood out of the body at a convenient location, preferably prior to blood passing through the kidneys, and passing it through a tube or tubes with the MIM fixed to blood contacting surfaces, carriers, or similar structures. Alternatively, the tube could have an insert, such as a planar or helical insert, which has the MIM attached or integrated in a composite fashion, or the MIM could form part or the entirety of the insert. Yet another possibility includes inclusion of supports such as regular or irregular beads or particles, either consisting of or coated with the MIM, or with the MIM being part of a composite, through which the blood passes.

In accordance with certain embodiments of the present invention, the blood flows over or around the matrix under conditions that cause less stress and mechanical damage as compared to conventional filtration or centrifuging. Also, in accordance with particularly useful aspects of the present invention, the sole component removed from the blood is the toxic contrast agent. By removing the contrast agent, this invention renders prophylactic measures used to protect the kidneys less necessary and in some cases unnecessary.

The detection of small traces of contrast agents in biological fluids relies on the existence of molecular recognition elements capable of selectively binding analytes with high affinity, selectivity, and capacity. Molecular recognition elements that are therefore typically used are proteins such as e.g., polyclonal, monoclonal, or recombinant antibodies, enzymes, receptors, oligo-nucleotides, etc. Besides their main advantage of high selectivity and association constants for the specific interaction process, such biological receptors have significant disadvantages: (i) they are difficult to obtain and therefore usually expensive, (ii) they only work in a certain—usually narrow—range of physical/physiological parameters including temperature, pressure, ionic strength, pH, etc., (iii) they usually require operation in aqueous buffers, and (iv) they are usually of limited applicability under harsh conditions. In addition to their poor stability, they are difficult to regenerate after one binding event. Moreover, specificity to the desired target analytes cannot always be generated due to natural limitations; generation of antibodies to antigens is frequently difficult owing to the small molecular mass and limited chemical complexity.

Accordingly, there is a need for alternative methods for the removal of contrast agents. The method should preferably be robust, specific, reliable, re-useable, of high binding capacity, and useable/producible/scalable on an industrial scale.

Molecular imprinting is a technique, which creates a polymer (or similar) matrix with binding sites for specific molecules based on a combination of recognition mechanisms including size, shape, and functionality. This technique is widely used in the field of biomimetic chemistry to develop duplications of binding sites of biological binding entities, and can also be called a synthetic receptor technology analogous in function to biological binding matrices.

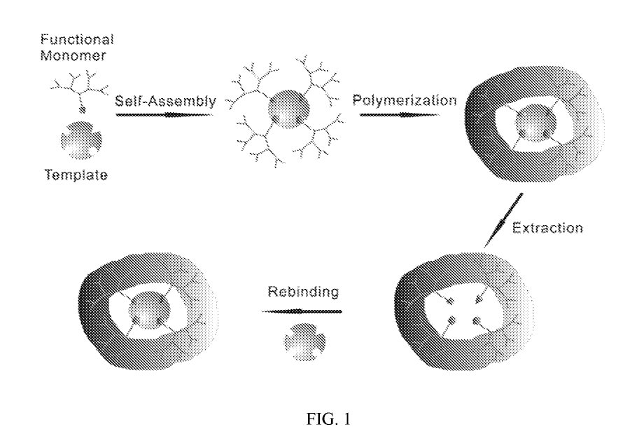

Molecular imprinting involves mixing a functional monomer capable of subsequent co-polymerization into a matrix, and the target molecule in solution and facilitating arrangement/binding of the functional monomer to the print molecules with a variety of possible interactions. After adding a cross-linking agent, a reaction is initiated via physical or chemical means inducing co-polymerization of the monomer and the cross-linker into a matrix. Then, the print molecules are removed by a variety of extraction processes, thereby leaving “molds” (a.k.a., binding sites complementary in shape, size, and functionality to the target/template molecule) in the matrix that can later entrap/re-recognize the “target” molecule (a.k.a., the print molecule) (see FIG. 1). Each “mold” or cavity can be configured to capture the entire molecule or a portion thereof, e.g., a terminal end or a (or several) functional group(s). Also, the matrix can physically trap the target molecule, and can optionally employ a wide variety of binding types including but not limited to ionic, electrostatic, covalent, hydrogen, or van der Waals binding. By creating such a matrix specifically tailored for radiographic contrast media, it is possible to create a material that will selectively remove the contrast media from the blood stream with minimal or no effect on other blood constituents.

Two main approaches to molecular imprinting have emerged to date, however, with a wide variety of modifications and combinations have been published: (i) the covalent approach pioneered by Wulff and Sarhan, and (ii) the non-covalent approach initially developed by Arshady and Mosbach. Covalent imprinting uses templates, which are covalently bound to one or more polymerizable functional monomer groups. After polymerization, the template bonds to the matrix are cleaved, and the functionality left in the binding site is capable of binding the target molecule by re-establishment of a covalent bond. The advantage of this approach is that the functional groups are only associated with the template site; however, only a limited number of compounds (including but not limited to e.g., alcohols (diols), aldehydes, ketones, amines, and carboxylic acids) can be imprinted with this approach. The most common covalent approach used for imprinting of templates bearing pairs of hydroxyl-groups (1,2- and 1,3-diol functionality) utilizes but is not limited to boronate esters. Monoalcohol templates can be imprinted by using e.g., but not limited to a boronophthalide-based monomer. While the well-defined stoichiometry associated with the covalent approach certainly has its merits, non-covalent imprinting based on non-covalent interactions such as but not limited to H-bonding, ion-pairing, and dipole-dipole interactions is dominating the literature, as this approach is readily adaptable and facilitates rapid synthesis, provides closer resemblance to the molecular recognition mechanisms of natural receptors, and benefits from the availability of substantial functional monomer libraries reported in literature.

Semi-covalent imprinting attempts to combine the advantages of the covalent and the non-covalent approach. As the template is covalently bound to a polymerizable functional monomer group, the functionality which is recovered after cleavage of the template should only be found in the binding site. However, re-binding takes place via semi- or non-covalent interactions. In stoichiometric non-covalent imprinting, the complex between functional monomer and template is strong enough to ensure that the equilibrium lies well on the side of the complex, therefore ensuring that it retains its integrity during the polymerization process; this can usually be ensured if the association constant (Kₐ) for the template-monomer interaction is ≧10³ M⁻¹.

Conventionally, MIMs are prepared as bulk polymer monoliths followed by mechanical grinding and sieving, thereby providing small (milli- to micrometer-sized) particles. Whereas the materials obtained through this somewhat inelegant, but straightforward method remains useful for many applications, methods for preparing imprinted polymers in more defined physical formats have attracted substantial interest. The synthesis of imprinted polymer beads for binding assays and separation resins can frequently be realized by adapting synthetic routes for generating conventional (non-imprinted) polymeric micro- and nanospheres. Furthermore, in sensor technology and for the proposed catheter applications the preferred MIM format is a thin layer or membrane produced by polymerization within a mould or directly at a substrate surface. Finally, grafting approaches have also been applied, and electropolymerization procedures have been used to build up layers of e.g., acrylamide-based MIMs at ISFET (ion-sensitive field effect transistor) surfaces. Alternatively, a MIM material shaped as regular or irregular particle may be incorporated in thin layer or membrane serving as a structural scaffold coated at the device surface.

As a prerequisite for developing a MIM, the print molecule should be soluble in the solvent used for establishing the initial imprint solution, and should provide suitable functional groups for interaction with the functional monomers to ensure stable complexation. The structure and chemical characteristics of the template usually determines the nature of the imprinting approach. Functional monomers are generally selected for strong interactions with the template during pre-arrangement in solution prior to the polymerization. So far, there is no general protocol or rational design scheme aiding the design of molecularly imprinted media. Hence, usually empirical knowledge is the starting point for the decision which ingredients (and at which ratios) should be selected. For example, if the target molecule can form complexes with certain metal ions, metal chelating functional monomers may be suitable. Methacrylic acid can provide ionic interactions to basic functional groups within the template. If hydrophobic interactions are desired, the imprinting solvent can be adjusted accordingly to enhance the binding strength of the pre-assembled complex.

To design MIMs for extracting contrast agents, the print molecule may be selected from ionic and non-ionic iodinated radiocontrast agents. Ionic radiocontrast agents include, but are not limited to, diatrizoate, metrizoate, and ioxaglate. Non-ionic radiocontrast agents include, but are not limited to, iohexyl, iodixanol, ioversol, iopamidol, iopamidol, ioxilan, and iopromide.

There is vast choice of available monomers with different functional groups for non-covalent imprinting. These can be basic or acid, permanently charged, hydrogen bonding, hydrophobic, metal coordinating, etc. It is of importance that the functional monomer strongly interacts with the template prior to polymerization, since the structure of the resulting assemblies in solution presumably defines the subsequently formed binding sites. By stabilizing the monomer-template assemblies, it is possible to increase the number of imprinted sites, and therefore tailor the binding capacity of the resulting MIM. At the same time, the number of non-specific binding sites will be minimized. The functional monomer should interact with the template and form e.g., but is not limited to hydrogen bridges or other non-covalent interactions. Exemplarily, but not limited to, acrylate and derivatives (acrylamide, methacrylate, etc.) or styrene can be used as functional monomer. Polyacrylate-based matrices have the advantage that the polymerization is easy to initiate (UV radiation or heat), and finalized within few hours. Besides, the solubility is of crucial importance for the practical usefulness of the functional monomer. Acrylates are soluble in a wide range of solvents, and at ambient temperature and pressure. Functional monomers used in the molecular imprinting process can include, but are not limited to, acrylic acid, acrylamide, agarose, methacrylic acid, trifluoro-methacrylic acid, 4-vinylbenzoic acid, itaconic acid, 4-vinylbenzyl-iminodiacetic acid, 2-acrylamido-2-methyl-1-propane sulphonic acid, 1-vinylimadazole, 2-vinylpyridine, 4-vinylpyridine, N,N-diethylaminoethyl methacrylate, aminostyrene, vinyl pyrrolidone, vinylimidazole, 4(5)-vinylimidazole, 3-acrylamidopropyltrimethylammonium chloride, styrene, 2-(methacryloyloxy)ethyl phosphate, and mixtures thereof.

The cross-linking monomer is responsible for mechanical and thermal stability of the polymer. It should be able to sufficiently “freeze” the pre-polymerization complex in its position. On the other hand, it should provide sufficient porosity to easily release the template after the imprinting process, and give access to the target for rebinding. Hence, template leaking from the polymer should be low, and the polymer backbone should provide sufficient micro-, macro-, and meso-channels for the target to rapidly diffuse to the binding site. Furthermore, the cross-linking polymer is another key component for achieving selectivity, due to its structure-stabilizing effect on the resulting binding cavities. Cross-linkers can be selected from the group comprising, but not limited to, 4-divinylbenzene, N,N′-methylene-bisacrylamide, N,N′-phenylene-bisacrylamide, 2,6-bisacrylamidopyridine, ethylene glycol dimethacrylate, pentaerythritol triacrylate, pentaerythritol tetraacrylate, trimethylolpropane trimethacrylate, and mixtures thereof.

The solvent plays an important role in the generation of MIMs. Three major characteristics affect the choice of the solvent to be used: (1) its effect on the structure of the MIM in its final format, (2) that it has to dissolve template, monomer, cross-linker, and polymerization initiator, and (3) that the solvent controls the porosity and the polymer morphology, and governs the strength of non-covalent interactions responsible for the integrity of the pre-polymerization complex.

Ideal solvents for molecular imprinting typically have a very low dielectric constant such as e.g., toluene or chloroform (i.e., at 20° C. below 40, preferably below 20, especially below 10). MIMs imprinted with non-polar solvents usually exhibit the best recognition properties for an analyte. Non-polar solvents do not disturb the interaction between template and monomer in contrast to e.g., water, which is highly polar and strongly associates with most templates. However, imprints synthesized in aqueous solution are of substantial interest for subsequent application in water-based media. Consequently, in most cases the choice of solvent is predominantly dependent on the solubility of the print molecule and the additional imprinting building blocks. For example, electrostatic interactions of pre-polymerization complexes are sensitive to the presence of polar protic solvents. The degree of dissociation of ion-pairs is strongly dependent on the solvent. In solvents of high dielectricity (>40), such as e.g., water, ion association is only noticeable in solutions at very high concentrations. On the other hand, practically no free ions are found in solvents of dielectric constants<10 such as e.g., toluene and dichloromethane. In solvents of intermediate dielectric constant such as e.g., acetonitrile or acetone, the degree of ion association is dependent on additional factors, such as size and charge distribution of the ions.

Porogens (a.k.a., pore forming solvents) can be, but are not limited to, toluene, acetonitrile, chloroform, methanol, ethanol, dichloromethane, water, and mixtures thereof.

Materials should be selected that are biocompatible or will yield a biocompatible product after polymerization, and such materials are known to one skilled in the art, especially those with knowledge of dialysis and apheresis.

Alternatively to conventional imprint materials, a heat-sensitive compound such as agarose, gelatin, heat sensitive hydrogels, or thermoplastic polymers can be used to form the “molds” of the functional monomers without the polymerization step. The substance is heated until liquefied with the print molecule added and then cooled to form the selective matrix.

In order to facilitate (initiate) the cross-linking (polymerization) of the monomer-print molecule admixture to form the imprinted medium, heat, radiation, or chemical initiation can be utilized depending on the selected materials. A number of different photo- and/or thermolabile initiators have been used in literature, with the most common being, but not limited to, 2,2′-azobis-(2,4-dimethylvaleronitrile) (ABDV) and azobis-(isobutyronitrile) (AIBN). The azobisnitriles are decomposed by heat (ABDV: 40° C.; AIBN: 60° C.) or UV light resulting in N₂ and two meta-stable radicals. The polymerization process involves three principal phases: (i) initiation, (ii) propagation, and (iii) termination. The ability of O₂ to accept an additional electron from the radicals leads to premature chain-termination. Hence, the monomer mixture is usually sparged with N₂ or He prior to polymerization. Moreover, templates with antioxidant properties, such as certain phenolic compounds, or molecules with long conjugated p-electron systems, may act as scavengers to inhibit polymerization and/or potentially be covalently incorporated into the MIM. Electrostatic interactions are anticipated to be mainly responsible for generating the MIM binding sites. Hence, it has been assumed that polymerization at low temperatures would be beneficial for the imprinting process. It was found that the separation factor a (Capacity factor: k′=(t−t₀)/t₀, t and t₀ is the retention time of analyte and void marker, respectively; separation factor: α=k′ (template)/k′ (template analogue) is the ratio of the capacity factors) increases with decreasing temperature of polymerization using AIBN at 0° C. On the other hand, unchanged selectivity by thermal polymerization was observed. It was found that by finishing photo initiated polymerizations with heating (120° C., 24 h) higher saturation capacities and improved chromatographic separation performance of the MIM is obtained.

Besides the right choice of monomer, cross-linker, solvent, and template to obtain useable MIMs of high selectivity, the ratio “template:functional monomer:crosslinker” must be determined. This ratio should be optimized to form specific binding sites for the template molecule within the MIM. The amount of the functional monomer should be, but is not limited to, 2 to 4 times the number of functional sites provided by the template molecule. A ratio of functional monomer:cross-linker of 1:5 is generally used (but not limited to this ratio) for ensuring the rigidity of the binding sites. For example, if the cross-linker has more than two vinyl groups, less cross-linker may be used. As an example, but not limited to, a typical ratio for MIMs based on MAA-EDMA (ethyleneglycol dimethacrylate) or 4VP-DVB (4-vinylpyridine divinylbenzene) copolymers is 1:8:40, and 1:4:4 for MAA-TRIM (trimethylolpropane tribethacrylate)co-polymers.

Selectivity of the imprinted polymer depends on the orientation of the functional groups inside the binding cavities, and the shape of the cavities. Selectivity increases with the number of binding interactions. First of all, during polymerization the interaction between binding site and template needs to be stable. The template should be extracted after polymerization under mild conditions and as complete as possible thereby avoiding template leaching at a later stage. For rebinding experiments and practical application, the equilibration with substrate molecules should be rapid and reversible. For example, it has been shown that non-covalent imprinting based on acrylic acid requires a fourfold excess of binding sites to ensure appropriate selectivity. Only approximately 15% of the cavities show re-uptake of a template under these conditions; the remaining approximately 85% are irreversibly lost for re-binding template, probably because of shrinking of the cavities. In general, the design depends on the desired application of the matrix.

After the matrix has solidified, the print molecule is extracted from the matrix by diffusion, chemical reaction, extraction, or other methods known in the art.

As an example, but not limited to these quantities or constituents, 1 mmol of iodixanol and 8 mmol of MAA are dissolved in a porogen (e.g., but not limited to, acetonitrile or acetone) followed by the addition of 40 mmol of EGDMA or DVB. After addition of AIBN (initiator, 2 wt. % of the total amount of monomer used), the solutions are sonicated and deoxygenated with nitrogen for 5 min, and then thermally polymerized at 60° C. for 24 h. The resulting bulk polymers are ground, and then wet-sieved/collected with acetone through a 25 μm sieve. The fine particles are removed by repetitive sedimentation in acetone. As a control, non-imprinted bulk polymers are prepared by exactly the same synthetic route in absence of the template molecule. To characterize the binding specificity of the resulting MIMs, the MIM particles as well as the control polymer particles may be suspended in acetone and packed e.g., into 150×4.6 mm stainless steel high-performance liquid chromatography (HPLC) columns with a slurry packer using acetone as the packing solvent, where they serve as stationary phase. Chromatographic analysis on their selective retention behavior may then be performed using a HPLC system equipped with e.g., a UV-Vis diode array detector, or another type of detector (e.g., but not limited to fluorescence detector, evaporate light scattering detector, etc.) indicating the elution of chromatographic peaks at the end of the separation path. Structural template analogues may be used to determine the selectivity of the MIM. Thereby, baseline separation of the template from its structural analogues in the resulting chromatogram is desired.

To study the binding capacity of the MIM, e.g., but not limited to, a 50 mg amount of the MIM or the control polymer (CTL), respectively, may be packed e.g., but not limited to, into 1 mL solid phase extraction (SPE) syringe barrels and capped with fritted polyethylene disks at the top and at the bottom. Prior to each use, the columns are conditioned with the following solvents (in order): methanol-acetic acid (4:1; v/v), methanol, methanol-2 M NaOH (1:1; v/v), methanol-water-acetic acid (18:1:1; v/v/v), methanol, and acetonitrile or water followed by the loading solvent. SPE syringe barrels containing e.g., but not limited to, 50 mg of C₁₈ or any other conventional sorbent may used for control experiments. The C₁₈ cartridges are conditioned successively with 10 mL of methanol and 10 mL of distilled water. The extraction is performed using a 12 port vacuum manifold. The application and elution solvents are drawn through the syringe barrels into vials, where 500 μL aliquots of the application solvent and 250 μL aliquots of the elution solvents are collected. The binding capacity of the MIM SPE cartridge is determined by recovery measurements of the template from the MIM cartridge and the CTL cartridge. After the columns are conditioned, 8 μg/mL template sample is applied in 0.5 mL aliquots onto the 1 mL syringe barrels packed with 100 mg MIM or CTL, respectively. The columns are washed with 2.5 mL of acetonitrile and eluted with 6 mL of methanol-acetic acid (7:1, v/v). The collected aliquots from the washing step (0.5 mL) are then directly analyzed by e.g., but not limited to, HPLC. The collected aliquots from the elution step (1 mL) may be dried under nitrogen and re-dissolved in 1 mL of the mobile phase. A C₁₈ column may be used in an isocratic elution with water-acetonitrile (1:1, v/v) as the eluent, and a flow rate of 1 mL/min. A typical capacity test results in a data matrix comparing the amount of template (usually in μg) applied to the MIM vs. the control material (CTL) in terms of the template amount loaded, how much remains in the material by sorption and after washing, how much is present after elution, and how much of the loaded template is finally recovered (in μg or %), and provides a direct performance comparison of the MIM against the control material.

A MIM network designed to recognize radiocontrast agents in the blood stream should function well in aqueous solution. However, the use of water as a solvent during molecular imprinting has been one of the limitations of this technique. Because water disrupts the hydrogen bonds formed between monomers and template recognition properties of networks prepared in organic solvents are stronger. However, it has been suggested that a number of weak interactions acting in a concerted fashion including e.g. but not limited to, hydrophobic forces, etc. can provide stronger interactions in aqueous media. In addition, other factors such as the ease of diffusion of analytes in and out of the polymer network also become important in real applications. Therefore, an easy binding/non-binding template switching method is desirable. Since the MIMs may be incorporated in the blood stream it is preferable if there are no harsh conditions to remove the template after re-binding, unless the MIM is used in a disposable fashion.

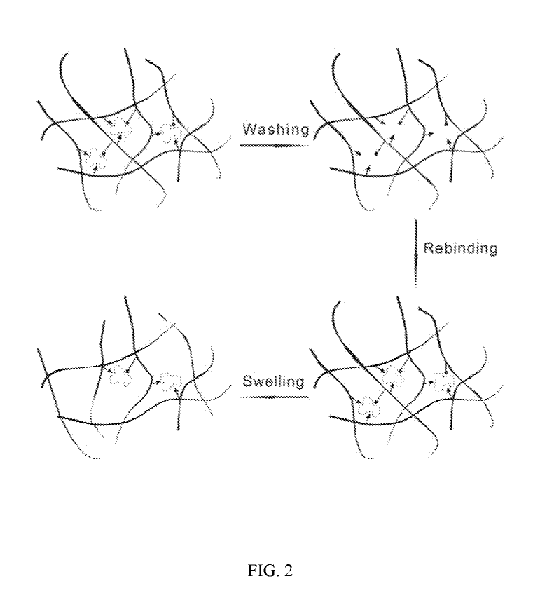

For these purposes, imprinting within e.g., but not limited to, hydrogels is suggested, as they can be prepared in aqueous solution, and can be switched “on and off” by external stimuli such as, but not limited to, pH, temperature, ionic strength, binding, etc., which modify their swelling behavior, as illustrated in FIG. 2, thereby benefitting either binding or release of the target.

However, proper tuning of the template-functional monomer interaction should be done to enhance binding and achieve selective recognition in aqueous media. There are many examples of such systems in nature. A good example is protein-ligand binding systems, which comprises dynamic binding functions in aqueous solutions. The macromolecular architecture of molecular imprinted hydrogels must be designed with an effective imprinting structure and proper rigidity to produce adequate specificity.

Alternatively, surface imprinting strategies provide substantial advantages for recognition of biologically relevant molecules or large molecules such as radiocontrast agents: the binding sites are more readily accessible at the surface of the imprinted matrix, and the mass transfer and binding kinetics are less limited given that template re-binding only requires the presence of the molecule at or close to the recognition surface. The specificity of these surfaces is predominantly based on shape selectivity, and hydrogen bonding interactions, but not limited to these types of interaction. While the achieved selectivity to date is not utterly convincing, this promising strategy certainly has potential for further optimization utilizing different recognition chemistries for functional mapping of target molecules.

Thin molecularly imprinted layers or membranes can be cast as a layer at the blood contacting surfaces of a number of intra- or extracorporeal medical devices. In one embodiment, the MIM can be prepared on a conventional smooth, cylindrical catheter or, alternatively, another intraluminal device with a geometry that increases the surface area of the MIM exposed to the blood flow, thereby facilitating binding with high capacity. The imprinted material is preferably configured to maximize the surface area contacting the blood. Examples of surface geometries include, but are not limited to, helices, multi-lobed extrusions (e.g., star-shaped), and crenellations. The coating can be integrated with a functional catheter by providing a coating on the exterior of the catheter, its interior lumen or at both surfaces. For example, the matrix can be applied to the guide catheter used to inject contrast agent into the coronary arteries. Alternatively, a separate intraluminal device can be provided and inserted into another location in the vasculature to remove the contrast agent prior to the kidneys. In another embodiment, the MIM material itself can provide the structural support being shaped into a catheter-like device. In yet another embodiment the MIM may be incorporated into a membrane or coating material applied to the surface of the device, thereby forming a composite material.

In another embodiment, the blood can be passed extracorporeally through a structure where the blood contacting surfaces are covered with MIM. Thus, the removal of the contrast agent would occur outside the body and without damaging the blood or extracting electrolytes as happens with dialysis or other vital blood constituents. In this example, the blood could pass through regularly shaped beads, irregularly shaped particles, fibers, or sheets, beads, or particles coated with the MIM, being a composite with MIM, or being made from MIM, in order to maximize the surface area available for contacting blood. Dialysis and apheresis methods are known to those of ordinary skill in the art and can be adapted to operate in accordance with some aspects of the present invention. A system such as that described in U.S. Patent Application Publication No. 2006/0030027, the contents of which are hereby incorporated by reference, may also be applicable to certain embodiments of the present invention

In a further embodiment, the body fluid contemplated is one of lymphatic and cerebral/spinal fluids, or urine. Just as radiographic contrast is injected into the blood stream to image blood vessels, it can also be injected into the cerebral/spinal fluid (CSF), lymph, or urine in order to image respectively, the spinal column (or cranial space), the lymphatic system, or the urinary tract. The contrast agent can then make its way into the blood and be transported to the kidneys or in the case of the urinary tract, migrate directly to the kidneys (via retrograde flow in the ureter). This contrast agent which is transported to the kidneys would have the same nephrotoxic potential as discussed previously. The MIM technology described herein can be used to either capture the contrast agent in the blood or alternatively directly from the lymph, the CSF, or the urine. This could be accomplished either by directly inserting the MIM into the appropriate anatomy (for example an appropriately sized MIM coated catheter inserted into a lymphatic duct, the spine, or the bladder) or by withdrawing the appropriate fluid out of the body and passing it through a MIM capture media prior to return the fluid to the body.

What is claimed is:

1. A method for removing radiocontrast agent from a patient comprising:
contacting body fluid of a patient with a molecularly imprinted medium (MIM) wherein said body fluid comprises a radiocontrast agent and said MIM is capable of selectively capturing the radiocontrast agent thereby facilitating removal of the radiocontrast agent from the body fluid of the patient.

2. The method of claim 1 wherein the radiocontrast agent comprises an ionic radiocontrast agent selected from the group consisting of diatrizoate, metrizoate, ioxaglate and mixtures thereof.

3. The method of claim 1 wherein the radiocontrast agent comprises a non-ionic radiocontrast agent selected from the group consisting of iohexyl, iodixanol, ioversol, iopamidol, iopamidol, ioxilan, iopromide, and mixtures thereof.

4. The method of claim 1 wherein the body fluid is selected from the group consisting of blood, lymphatic fluids, spinal fluids, urine, and mixtures thereof.

5. The method of claim 1 wherein the body fluid is contacted with the MIM extracorporeally.

6. The method of claim 5 wherein the method comprises:
removing the body fluid from the patient and contacting the body fluid with the MIM to extract the radiocontrast agent and produce a treated body fluid with a lower concentration of radiocontrast agent.

7. The method of claim 6 wherein said method further comprises returning treated body fluid to the patient.

8. The method of claim 6 wherein the body fluid comprises blood.

9. The method of claim 8 wherein said method further comprises returning the treated blood to the patient.

10. The method of claim 1 wherein the body fluid is contacted with the MIM intracorporeally.

11. The method of claim 10 wherein the method comprises contacting the body fluid with a device comprising the MIM.

12. The method of claim 11 wherein the device is at least partially coated with the MIM.

13. The method of claim 11 wherein the MIM is part of a coating at least partially covering the device.

14. The method of claim 11 wherein the MIM forms at least a part of the structure of the device.

15. The method of claim 11 wherein the body fluid comprises blood.

16. A device for removing radiocontrast agent from a patient comprising:
a molecularly imprinted medium (MIM) wherein said MIM reversibly binds to a radiocontrast agent; and
a support.

17. The device of claim 16 wherein the MIM forms at least a part of the support.

18. The device of claim 16 wherein the support comprises a catheter.

19. The device of claim 18 wherein the MIM is coated on the catheter.

20. The device of claim 18 wherein the MIM is part of a coating coated on the catheter.

21. The device of claim 18 wherein the MIM forms at least a part of the catheter.

22. The device of claim 16 wherein the support is selected from the group consisting of beads, particles, fibers, sheets, and combinations thereof.

23. The device of claim 22 wherein the support is coated with the MIM.

24. The device of claim 22 wherein the support is coated with the MIM being part of a coating.

25. The device of claim 22 wherein the MIM forms at least a part of the support.
